(19) United States
(12) Patent Application Publication
(10) Pub. No.: US 2025/0272698 A1
(43) Pub. Date: Aug. 28, 2025

(54) Systems And Methods For Verifying Authenticity Of Pharmaceuticals

(71) Applicant: Robert Hoose, Irvine, CA (US)

(72) Inventor: Robert Hoose, Irvine, CA (US)

(21) Appl. No.: 18/952,056

(22) Filed: Nov. 19, 2024

Related U.S. Application Data
(63) Continuation-in-part of application No. 18/586,077, filed on Feb. 23, 2024.

(51) Int. Cl. G06Q 30/018

(52) U.S. Cl. CPC G06Q 30/0185

(57) ABSTRACT

Systems and methods are presented for invalidating a good from a registry of confirmed authentic goods. As example, a user has an injectable pen with a unique identifier, and the user believes the injection is Ozempic™. The pen is similar to other pens (e.g., other pens having GLP-1 receptor agonists). The user accesses a database having a status of unique identifiers for each pend in the class of pens (e.g., injectable pens) and queries the unique identifier of the pen. The query returns status of the unique identifier in the database (e.g., invalid or valid), and enters the status as invalid after the query. If the user accessed the database anonymously, the status of the pen remains invalid after the query. If the user provides authenticated credentials to access the database, the user may return the status of the pen to the pre-query status (e.g., valid).

This application is a continuation in part of and claims the benefit of priority to U.S. Ser. No. 18/586,077 filed Feb. 23, 2024, which is incorporated in its entirety by reference herein.

### FIELD OF THE INVENTION

The field of the invention is consumer protection from mistaken goods.

### BACKGROUND

The background description includes information that may be useful in understanding the present invention. It is not an admission that any of the information provided herein is prior art or relevant to the presently claimed invention, or that any publication specifically or implicitly referenced is prior art. All references are incorporated herein in their entirety.

Validating the identity of goods, that a good is in fact what the user expects it to be, is challenging for goods having similar or identical appearance. Mistakeable goods can harm the user or legitimate seller alike. A seller of quality goods can develop a poor reputation if imitation goods of lesser quality are sold as the seller's own. A user likewise suffers where counterfeit goods are passed-off as authentic goods at great cost.

Attempts have been made to solve problems related to authenticity of goods. For example, U.S. Pat. No. 7,810,726 to de la Huerga teaches a unique code can be affixed to an item and stored in verification database. Before an item is sold, the code from the product is verified against the verification database to demonstrate authenticity. Further, U.S. Pat. No. 11,373,192 to Peckover teaches unique random numbers or codes are generated, stored, recalled, and imprinted on each item, for example each individual pill in a bottle is uniquely identified. A pointer links the unique code on the item to a value in a database validating the item.

Identifying features are also known to be used to verify authenticity of goods. U.S. Pat. No. 8,712,163 to Osheroff teaches imaging individual pills to identify and detect counterfeit goods. Different optical features of a good (e.g., shape, size, contrast, space between reference points, codes, patterns, etc.) can be used in a two step process, with identifying a primary marker prompting identifying a secondary marker. Likewise, U.S. Pat. No. 9,569,650 to Hanina et al. teaches labeling items with fractal patterns and verifying points in the fractal pattern to validate authenticity.

Thus, authentication of products or items to combat sale or distribution of counterfeit or fraudulent goods have been attempted. However, in spite of this mistakeable goods that are easy to imitate flood the marketplace with at times disastrous effects. The opiate epidemic in the United States is fueled by deadly fentanyl based drugs that are mistakeable for less harmful opiate pharmaceuticals.

Rather than focusing on proving that a pill or item is authentic, there remains a need for methods, devices, databases, and systems to invalidate the authenticity of the item and safeguard the public.

### SUMMARY OF THE INVENTION

The inventive subject matter provides databases, apparatus, systems, and methods of marking, signaling, or identifying a pill or consumable as invalid, unauthentic, counterfeit, potentially dangerous, or deadly. A method is disclosed for marking one or more consumables as invalid in a database. The one individual consumable is associated with a class of consumables and has a unique identifier. For example, the consumable could be a drug from the class of opiates or GLP-1 receptor agonists.

A user with access to the consumable (e.g., user has physical access, user knows where and how to access, access to the unique identifier of the consumable, etc.) accesses the database, which includes information about the class of consumables and the individual consumable. The user submits a query to the database that includes the unique identifier of the consumable. As a result of the query, a status of the consumable is marked as invalid in the database. Viewed from another perspective, by submitting the unique identifier in a query to the database, the database updates the status of the unique identifier as invalid. The user receives a response from the database showing whether the consumable was a valid or invalid member of the class of consumables before the user submitted the query. Subsequent queries of the specific consumable to the database show that specific consumable is invalid.

Methods of invalidating a specific item are contemplated. The specific item is one of many of items in a class of items, and can be individually identified or distinguished from the class of items by a unique identifier. A user accesses a database having information regarding the class of items. The user submits a query to the database that includes the identifier. The query (i) retrieves information relating to the specific item from the database and (ii) adds data regarding the specific item or the identifier to the database. The user receives a response from the database regarding a status of the specific item as a member of the class of items, for example whether the specific items is a valid or an invalid member of the class of items.

Further methods of invalidating a specific item in a database are contemplated. The specific item has a unique identifier that distinguishes the specific item from other similar items associated with a class of items. A user accesses the database having information regarding the class of items and members of the class of items. The user submits a query to the database that includes the unique identifier of the specific item. The specific item has a pre-query status in the database, with respect to submission of the query. The query retrieves information related to the specific item or the unique identifier, marks a status of the specific item as invalid in the database, or both. The user receives a response from the database indicating the pre-query status of the first item, for example whether the pre-query status is as a valid or invalid member of the class of items.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts a method of the inventive subject matter.

### DETAILED DESCRIPTION

Systems, devices, databases, and devices are contemplated for marking a member of a class of items (e.g., one pill among many similar or mistakeable pills) as invalid, preferably regardless of previous validity of the item.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

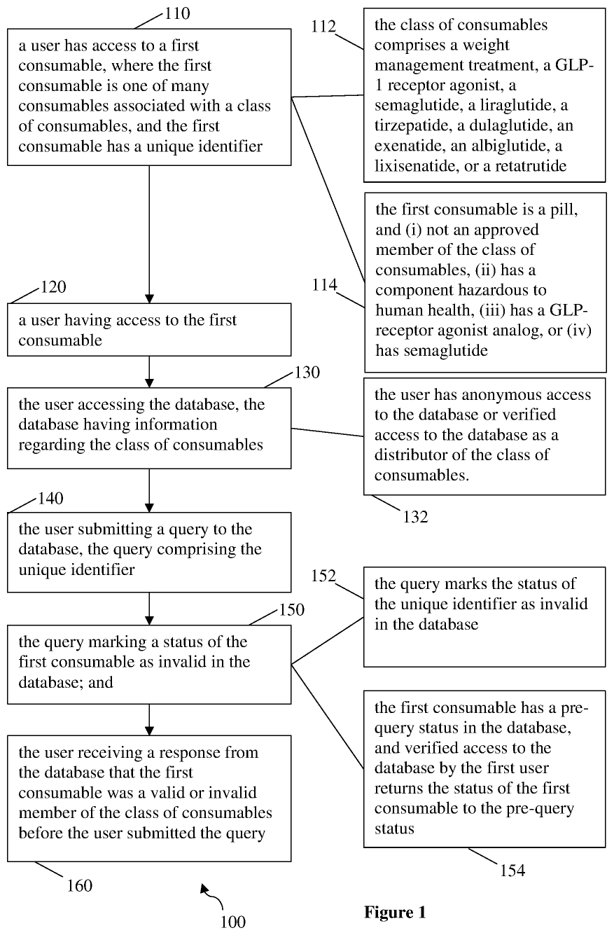

FIG. 1 depicts flowchart 100 of a method of the inventive subject matter. Steps 110, 120, 130, 140, 150, and 160 proceed in order as depicted. Optional features 112 and 114 can be used during step 110 and subsequent steps. Optional feature 132 can be used during step 130 and subsequent steps. Optional features 152 and 154 can be used during step 150 and subsequent step 160.

The inventive subject matter provides databases, apparatus, systems, and methods of marking, signaling, or otherwise identifying to a user that an item is not what it appears to be (e.g., a similar item, an inferior alternative, a dangerous substitute, a low quality replacement, etc.). For example a user may believe a specific pill is an authentic or valid pill of OxyContin™, when it is actually a dangerous or deadly pill including fentanyl. A mistake between an authentic OxyContin™ pill and a pill appearing similar but having fentanyl can be deadly for the user. To prevent users from mistaking the authenticity of items such as pills, methods are disclosed for marking one or more consumables such as pills as invalid in a database.

A specific consumable item has a unique identifier (e.g., code, label, mark, pattern, signal, electromagnetic signature, etc.) and is associated with a class of consumables. For example, the consumable item may be a pharmaceutical or therapeutic from a class of pharmaceuticals or therapeutics (e.g., opiates, GLP-1 receptor agonists etc.), food or drink from a brand or class of food or drink, or recreational drug or composition from a class of recreational drugs or compositions (e.g., recreational cannabis, compositions having cannabis, nicotine, etc.). Non-consumable items are also contemplated, such as luxury goods, clothes, jewelry, accessories, electronics, and other consumer goods or products.

A user with access to the consumable item or access to the unique identifier accesses a database having information about the class of consumables and the individual consumable item. The user submits a query to the database that includes the unique identifier of the consumable. After the query is submitted by the user, a status of the consumable is marked as invalid (or fake, unauthentic, ingenuine, fraudulent, counterfeit, tampered, stolen, etc.) in the database. Thus, after a user checks the authenticity of the consumable item by submitting the unique identifier as a query to the database, the database logs the status of the unique identifier as invalid or unauthentic for subsequent queries.

After submitting the query, the user receives a response from the database showing whether the consumable was a valid or invalid member of the class of consumables before the user submitted the query. Subsequent queries of the specific consumable to the database show that specific consumable is invalid.

The class of consumables can include pharmaceuticals or drugs, for example including Schedule I-V substances classified by the Drug Enforcement Administration under the Controlled Substances Act, an analog of a Schedule I-V substance, an opiate, or GLP-1 receptor agonists. The class of consumables can include foods, pills, tabs, powders, gels, gummies, slurries, liquids, tonics, tinctures, gases, vapors, etc. intended for human consumption.

Further consumables and items other than consumables can be marked as invalid or unauthentic in the inventive subject matter. For example, nutraceuticals, fat burners, weight loss pills, energy supplements, legal cannabis, prescription and over the counter drugs, and fungible consumable goods. Drugs can include antibiotic, pain management, opiates, anti-inflammatory, erectile disfunction, weight management or diet pills, etc. Preferred embodiments address classes of items where distinguishing between authentic, true members of the class and invalid, unauthentic members of the class is difficult for consumers and may result in bodily harm.

Further consumables considered applicable to the inventive subject matter include weight management, blood sugar, or diabetes treatments typically available as a drug or pharmaceutical, such as GLP-1 receptor agonists (incretin mimetics) including semaglutide (e.g., Ozempic™, Wegovy™, Rybelsus™, etc.) as well as liraglutide, tirzepatide, dulaglutide, exenatide, albiglutide, and lixisenatide. It is contemplated the inventive subject matter can be used to differentiate or otherwise identify valid, true, untainted, or untampered GLP-1 receptor agonists (and other consumables described herein) from false, imposter, unauthentic, counterfeit, tampered, tainted, or dangerous compositions, for example salt forms of one or more of the underlying ingredients.

GLP-1 receptor agonists are known in the art. See, e.g. Yao et al., “Comparative effectiveness of GLP-1 receptor agonists on glycaemic control, body weight, and lipid profile for type 2 diabetes: systematic review and network meta-analysis,” BMJ Jan. 29, 2024, PMC10823535; Tan et al., “Efficacy and Safety of Semaglutide for Weight Loss in Obesity Without Diabetes: A Systematic Review and Meta-Analysis,” J ASEAN Fed Endocr Soc Aug. 23, 2022, PMC9758543.

Further drugs, treatments, pharmaceuticals, or other consumables containing other glucose or glucagon related agonists are contemplated applicable to the inventive subject matter, for example agonists of glucose-dependent insulinotropic polypeptides or glucagon receptors such as retatrutide. See, e.g., Jastreboff et al., “Triple-Hormone-Receptor Agonist Retatrutide for Obesity—A Phase 2 Trial,” N Engl J Med 2023, 389:514-526.

Such consumables may be in pill or tablet format, or available as tinctures, injections, powders, gels, creams, oils, inhalants, patch, or other mediums and adapted for oral, buccal, sublingual, rectal, parenteral, topical, or inhalational administration, or otherwise providing passive diffusion, facilitated passive diffusion, active transport, or pinocytosis of the consumable or drug into the human body.

In preferred embodiments the consumable is a pill, and is either not an approved member of the class of consumables, includes a component hazardous to human health, includes a fentanyl analog, or includes fentanyl.

While the user may identify themselves when querying the database, in preferred embodiments the user can choose to remain anonymous, and can access the database anonymously. It is critical that when the user queries a unique identifier anonymously, submission of the anonymous query marks the status of the unique identifier or the consumable as invalid in the database. Thus, regardless of whether the specific consumable is a valid member of the class or not, all queries of the unique identifier submitted after the anonymous user will identify the specific consumable as invalid.

Where the user chooses to identify themselves, they may further verify their access to the database, for example where a verified distributor of the class of consumables has verified access to the database. Preferably where the user has verified access to the database, querying the unique identifier of a consumable does not cause the unique identifier or consumable to be marked as invalid or unauthentic in the database. Viewed from another perspective, where the user verified access to the database, the pre-query status of the unique identifier or specific consumable is unchanged by the query and the status is reported to the verified user.

Methods of invalidating a specific item are contemplated. The specific item is one of many of items in a class of items, and can be individually identified or distinguished from the class of items by a unique identifier. A user accesses a database having information regarding the class of items. The user submits a query to the database that includes the identifier, preferably anonymously. The query retrieves information related to the specific item from the database and, when the user anonymously submits the query, adds data regarding the specific item or the identifier to the database. The user receives a response from the database regarding a pre-query status of the specific item as a member of the class of items, for example whether the specific items is a valid or an invalid member of the class of items.

The class of items can be a consumable, such as a pharmaceutical, a generic pharmaceutical, a brand name pharmaceutical, an opiate pharmaceutical, a nutraceutical, a dietary supplement, or food or drink. The class of item can also be non-consumable, such as luxury goods, clothes, jewelry, accessories, electronics, or other consumer goods.

In some embodiment the item the user queries is (i) not an approved member of the class of items, (ii) comprises a component hazardous to human health, (iii) comprises a fentanyl analog, or (iv) comprises fentanyl. In such cases the inventive method identifies the item as invalid or unauthentic to the user and maintains designation in the database as invalid or unauthentic. Where the class of items is potentially hazardous to the user's health (e.g., a pharmaceutical or pill) and the query returns the specific item or unique identifier is invalid, a warning may further be delivered to the user warning the item may be deadly if consumed.

While the user may query the database anonymously, the user may also submit identifying information when making the query. The user may also submit credentials or otherwise gain verified access to the database, for example where the user is a verified distributor of the class of items or is verified to provide the item to others. However, where the user has anonymous access to the database, a query submitted to the database with the unique identifier invalidates that unique identifier or specific item.

Further methods of invalidating a specific item in a database are contemplated. The specific item has a unique identifier that distinguishes the specific item from other similar items associated with a class of items. A user accesses the database having information regarding the class of items and members of the class of items. The user submits a query to the database that includes the unique identifier of the specific item. The specific item has a pre-query status in the database, with respect to submission of the query by the user. The query (i) retrieves information related to the specific item or the unique identifier, (ii) marks a status of the specific item as invalid in the database, or both. The user receives a response from the database indicating the pre-query status of the first item, for example whether the pre-query status is as a valid or invalid member of the class of items.

In some embodiments the user submits a credential to the database, for example before or after submitting the query. If credentials submitted by the user are valid and approved, the status of the specific item is returned from invalid to the pre-query status provided the time between submitting the query and submitting the credentials is not greater than 30 seconds, 1 minute, 2 minutes, or 5 minutes. Expiration of a time limit between the user submitting the query and submitting the credential prevents reverting the status to the pre-query status for that specific item. Such safeguards are advantageous where the item or class of items is an opiate pharmaceutical, and in such embodiments the response to the query further includes a warning that if the pre-query status is invalid, consuming the opiate pharmaceutical is deadly.

Systems are contemplated that use a database to record known authenticity of a pill or item until the authenticity is checked by an anonymous end user. After an anonymous user queries the status of the pill or item, the recorded authenticity recorded in the database is invalidated. While this may invalidate otherwise valid or authentic items or pills, it impedes a user from subsequently transferring such items or pills under the guise of being authentic as any subsequent query of authenticity will show the pill or item is invalid. The user is protected by such a system because if the pre-query status is authentic, the user knows the item or pill is safe for its intended use.

In some embodiments the database is specific to a certain class of items, e.g. specific to pills of OxyContin™, specific to pills of Viagra™, specific to pills having cannabinoid compositions, specific to items affiliated with a brand or authentic source, etc. It is contemplated that multiple databases, each specific to its own class of item, can be linked or queried by a single query from the user. It is further contemplated a database contains status of more than one class of items, for example containing status for OxyContin™ and Viagra™, or multiple classes of related items, for example containing status for OxyContin™, Vicadin™, Percocet™, and other related opiates, or classes otherwise related (e.g., apparel, accessories, pharmaceutical, nutraceutical, dietary supplements, etc.).

Such a system contemplates a validation or authenticity database for a class of items (e.g., pills) based on a unique identifier on each item associated with an authenticity indicator for each item in the database. Any user can query the database with the unique identifier, for example a pharmacist, ER personnel, a first responder, a gray or black-market seller or buyer, or a consumer. Before querying the authenticity database a user chooses between entering an identification of themselves, entering access credentials for themselves, or making the query anonymously. Providing access credentials returns a result from the database, for example confirming the item as valid, authentic, counterfeit, unknown, etc.

Choosing anonymous access automatically invalidates the authenticity of the database result (e.g., unique identifier or specific identifier is invalid), but returns the true, pre-query database result to the user. It is contemplated all queries after an anonymous query will show the item is invalid, while the first anonymous query will return whether the item is authentic or not. Invalidating the pill/drug with an anonymous query protects the end user from being known, and protects them from taking suspicious or unknown pills/drugs.

In some embodiments identifying data alone is enough to preserve the pre-query status of the item or the unique identifier in the database, while others require access credentials to preserve the pre-query status. Identifying data could be approved identification for authorized users (e.g., pharmacist license, pharmacy ID, etc.), or trackable/traceable identification data for gray/black market users (e.g., Driver's License, personal cell number or email with verification code, SSN, credit card information, location data from a smartphone, etc.). Providing identifying data could also alert or prepare first responders for potential emergencies where the queried item is invalid or deadly.

In embodiments where the database contains status specific to a certain type of item, the query will return the status of the item in the database (e.g., valid/authentic, invalid/unauthentic, or not found on registry (i.e., potential fraud or otherwise unauthentic)). Where the database is linked to other databases containing status of other specific classes of items, or the database includes status of multiple classes of items, the query will return the status of the item or the identity of the item, or both.

For example, the query may return the item status is “valid” and identity “OxyContin™” where the unique identifier was found in the database, had “valid” status, and had “OxyContin™” as the identity associated with the unique identifier. For instances where the unique identifier is not found in the database or linked database, the unique identifier may be added to the database with a status of invalid/unauthentic, an identity of unknown source, or both. In some embodiments where the unique identifier is not found in a database, no entries are further added and the return to the query is simply invalid/unauthentic.

Similarly, the query may return status “invalid” and identity “other” where the unique identifier was not found in the specific database queried but was found in a linked database (i.e., identity as “other” as found in a linked database). In some embodiments, it is preferred a query simply returns “invalid” where the status of the unique identifier is invalid, with an optional warning against deadly consequences of using or taking invalid items. In at least one embodiment, where the status of the unique identifier for the item is “invalid,” but the identity is registered as a dangerous or addictive substance in the database or linked database (e.g., known OxyContin™, known opiate, etc.), the query does not inform the user the identity of the item to prevent potential substance abuse or illicit trade of the item.

As used herein, and unless the context dictates otherwise, the term “attached to” and “coupled to” are intended to include both direct coupling (in which two elements that are coupled to each other contact each other) and indirect coupling (in which at least one additional element is located between the two elements). Therefore, the terms “attached to,” “coupled to,” “attached with,” and “coupled with” are used synonymously.

It should be apparent to those skilled in the art that many more modifications besides those already described are possible without departing from the inventive concepts herein. The inventive subject matter, therefore, is not to be restricted except in the spirit of the amended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . and N, the text should be interpreted as requiring only one element from the group, not A plus N, or B plus N, etc.

What is claimed is:

1. A method of marking a first consumable as invalid in a database, wherein the first consumable has a unique identifier and the first consumable is associated with a class of consumables, comprising:
a user having access to the first consumable;
the user accessing the database, the database having information regarding the class of consumables;
the user submitting a query to the database, the query comprising the unique identifier;
the query marking a status of the first consumable as invalid in the database; and
the user receiving a response from the database that the first consumable was a valid or invalid member of the class of consumables before the user submitted the query.

2. The method of claim 1, wherein the class of consumables comprises a weight management treatment, a GLP-1 receptor agonist, a semaglutide, a liraglutide, a tirzepatide, a dulaglutide, an exenatide, an albiglutide, a lixisenatide, or a retatrutide.

3. The method of claim 1, wherein the first consumable is a pill, and (i) not an approved member of the class of consumables, (ii) comprises a component hazardous to human health, (iii) comprises an analog of one of semaglutide, liraglutide, tirzepatide, dulaglutide, exenatide, albiglutide, lixisenatide, or retatrutide, or (iv) comprises semaglutide, liraglutide, tirzepatide, dulaglutide, exenatide, albiglutide, lixisenatide, or retatrutide.

4. The method of claim 1, wherein the user has anonymous access to the database or verified access to the database, wherein verified access is as a verified distributor of the class of consumables.

5. The method of claim 1, wherein the query marks the status of the unique identifier as invalid in the database.

6. The method of claim 1, wherein the user has anonymous access to the database.

7. The method of claim 1, wherein the user has verified access to the database, the first consumable has a pre-query status in the database, and wherein verified access by the first user returns the status of the first consumable to the pre-query status.

8. A method of invalidating a first item having a unique identifier, wherein the first item is one of a plurality of items associated with a class of items, comprising:
a user accessing a database having information regarding the class of items;
the user submitting a query to the database, the query comprising the identifier;
the query adding a data regarding the identifier to the database; and
the user receiving a response from the database regarding a status of the first item as a member of the class of items.

9. The method of claim 8, wherein the status is one of a valid or an invalid member of the class of items.

10. The method of claim 8, wherein the class of items is one of a consumable, a pharmaceutical, a generic pharmaceutical, a brand name pharmaceutical, a weight management pharmaceutical, a nutraceutical, or a dietary supplement.

11. The method of claim 8, wherein the first item is (i) not an approved member of the class of items, (ii) comprises a component hazardous to human health, (iii) comprises an analog of one of semaglutide, liraglutide, tirzepatide, dulaglutide, exenatide, albiglutide, lixisenatide, or retatrutide, or (iv) comprises semaglutide, liraglutide, tirzepatide, dulaglutide, exenatide, albiglutide, lixisenatide, or retatrutide.

12. The method of claim 8, wherein the user has anonymous access to the database or verified access to the database, wherein verified access is as a verified distributor of the class of items.

13. The method of claim 8, wherein the data invalidates the first item in the database.

14. The method of claim 13, wherein the user has anonymous access to the database.

15. The method of claim 8, wherein the user has verified access to the database, and the data does not invalidate the first item in the database.

16. The method of claim 8, wherein the user has physical access to the first item.

17. A method of invalidating a first item in a database, wherein the first item has a unique identifier and the first item is associated with a class of items, comprising:
a user accessing the database, the database having information regarding the class of items;
the user submitting a query to the database, the query comprising the unique identifier;
the query marking a status of the first item as invalid in the database, wherein the first item has a pre-query status in the database;
the user receiving a response from the database of the pre-query status of the first item, wherein the pre-query status is as a valid or invalid member of the class of items.

18. The method of claim 17, further comprising the user submitting a credential to the database, wherein submitting the credential returns the status of the first item from invalid to the pre-query status.

19. The method of claim 18, further comprising a time limit for the user to submit the credential, wherein expiration of the time limit prevents subsequent modification of the status of the first item.

20. The method of claim 17, wherein the first item is an opiate pharmaceutical, and the response comprises a warning that if the pre-query status is invalid, ingestion of the opiate pharmaceutical is deadly. CLAIMS Systems and methods are presented for invalidating a good from a registry of confirmed authentic goods. As example, a user has an injectable pen with a unique identifier, and the user believes the injection is Ozempic™M. The pen is similar to other pens (e.g., other pens having GLP-1 receptor agonists). The user accesses a database having a status of unique identifiers for each pend in the class of pens (e.g., injectable pens) and queries the unique identifier of the pen. The query returns status of the unique identifier in the database (e.g., invalid or valid), and enters the status as invalid after the query. If the user accessed the database anonymously, the status of the pen remains invalid after the query. If the user provides authenticated credentials to access the database, the user may return the status of the pen to the pre-query status (e.g., valid).
